(19) United States
(12) Patent Application Publication
(10) Pub. No.: US 2025/0054120 A1
(43) Pub. Date: Feb. 13, 2025

(54) SYSTEMS AND METHODS FOR IMAGE ANALYSIS

(71) Applicant: WUHAN UNITED IMAGING HEALTHCARE CO., LTD., Wuhan (CN)

(72) Inventor: Xing MING, Wuhan (CN)

(73) Assignee: WUHAN UNITED IMAGING HEALTHCARE CO., LTD., Wuhan (CN)

(21) Appl. No.: 18/927,875

(22) Filed: Oct. 26, 2024

Related U.S. Application Data
(63) Continuation of application No. PCT/CN2023/090657, filed on Apr. 25, 2023.

(30) Foreign Application Priority Data
Apr. 26, 2022 (CN) 202210443625.X

(51) Int. Cl. G06T 7/00; G06V 10/44

(52) U.S. Cl. CPC G06T 7/00; G06V 10/44

(57) ABSTRACT

The present disclosure provides methods and systems for image analysis. The methods may be implemented by a gateway device between an image management platform and an image analysis service platform. The methods may include obtaining a request for analyzing target image data stored in the image management platform. In response to the request, the methods may include generating an image analysis task with respect to the target image data. The image analysis task may include a storage address of the target image data in the image management platform. The methods may further include transmitting the image analysis task to the image analysis service platform to direct the image analysis service platform to acquire the target image data from the storage address and perform image analysis on the target image data.

### CROSS-REFERENCE TO RELATED APPLICATIONS

This application is a continuation of International Application No. PCT/CN2023/090657, filed on Apr. 25, 2023, which claims priority to Chinese Patent Application No. 202210443625.X, filed on Apr. 26, 2022, the contents of which are incorporated herein by reference.

### TECHNICAL FIELD

The present disclosure generally relates to image processing, and more particularly, relates to systems and methods for image analysis.

### BACKGROUND

Through data processing techniques (e.g., a cloud computing technique, a big data technique, a mobile Internet technique, etc.), an image management platform can realize the centralized storage, filing, sharing, etc., of regional image data and diagnostic reports. Users can use mobile devices to interact with the image management platform to retrieve, read, and share the image data and diagnostic reports.

Sometimes, the image management platform may send image data to a third-party image analysis service platform for performing image analysis on the image data. Recently, with the development of AI techniques, some AI image analysis service platforms have been developed to provide services like image classification, detection, identification, segmentation, etc., to the image management platform. At present, the imaging management platform needs to transmit the received image data to the Al image analysis service platform and direct the AI image analysis service platform to perform image analysis. However, information leakage may happen during the data transmission process, and a lot of computing resources and network bandwidth are required for image data transmission.

### SUMMARY

In an aspect of the present disclosure, a method for image analysis is provided. The method may be implemented by a gateway device between an image management platform and an image analysis service platform. The method may include obtaining a request for analyzing target image data stored in the image management platform. The method may include generating an image analysis task with respect to the target image data in response to the request. The image analysis task may include a storage address of the target image data in the image management platform. The method may further include transmitting the image analysis task to the image analysis service platform to direct the image analysis service platform to acquire the target image data from the storage address and perform image analysis on the target image data.

In some embodiments, the generating an image analysis task with respect to the target image data may include determining whether one or more features of the target image data satisfy a first condition; and in response to determining that the one or more features of the target image data satisfy the first condition, generating the image analysis task with respect to the target image data.

In some embodiments, the generating an image analysis task with respect to the target image data may include determining whether user permission corresponding to the request satisfies a second condition; and in response to determining that the user permission corresponding to the request satisfies the second condition, generating the image analysis task with respect to the target image data.

In some embodiments, the generating an image analysis task with respect to the target image data may include obtaining a plurality of candidate algorithms; determining a target algorithm from the plurality of candidate algorithms based on the target image data; and generating the image analysis task with respect to the target image data based on the target algorithm.

In some embodiments, the determining a target algorithm from the plurality of candidate algorithms based on the target image data may include, for each of the plurality of candidate algorithms, determining at least one mandatory requirement and at least one optional requirement with respect to an input of the candidate algorithm; selecting at least one candidate algorithm from the plurality of candidate algorithms, the target image data satisfying the at least one mandatory requirement of each selected candidate algorithm; and determining the target algorithm from the at least one selected candidate algorithm based on the target image data and the at least one optional requirement of each selected candidate algorithm.

In some embodiments, the determining a target algorithm from the plurality of candidate algorithms based on the target image data may include obtaining a corresponding relationship between the plurality of candidate algorithms and a plurality of reference feature vectors representing a plurality of sets of reference image data; determining a target feature vector representing the target image data; and determining the target algorithm from the plurality of candidate algorithms based on the target feature vector and the corresponding relationship.

In some embodiments, the determining a target algorithm from the plurality of candidate algorithms based on the target image data may include obtaining an algorithm determination model, wherein the algorithm determination model is a trained machine learning model; and determining the target algorithm based on the target image data using the algorithm determination model.

In some embodiments, the method may further include obtaining, from the image analysis service platform, an analysis abstract of an image analysis result of the target image data; and storing the analysis abstract into a storage component of the gateway device.

In some embodiments, the method may further include obtaining, from a user terminal, a second request for obtaining an image analysis result of the target image data; and transmitting the second request to the image analysis service platform to direct the image analysis service platform to transmit the image analysis result to the user terminal.

In some embodiments, the gateway device may be integrated into the image management platform.

In another aspect of the present disclosure, a system for image analysis is provided. The system may include a gateway device between an image management platform and an image analysis service platform. The system may include at least one storage device including a set of instructions; and at least one processor configured to communicate with the at least one storage device. When executing the set of instructions, the at least one processor may be configured to direct the system to perform operations. The operations may include obtaining a request for analyzing target image data stored in the image management platform. The operations may include generating an image analysis task with respect to the target image data in response to the request. The image analysis task may include a storage address of the target image data in the image management platform. The operations may include transmitting the image analysis task to the image analysis service platform to direct the image analysis service platform to acquire the target image data from the storage address and perform image analysis on the target image data.

In still another aspect of the present disclosure, a gateway device between an image management platform and an image analysis service platform is provided. The gateway device may include an obtaining module, a generation module, and a transmission module. The obtaining module may be configured to obtain a request for analyzing target image data stored in the image management platform. The generation module may be configured to generate an image analysis task with respect to the target image data in response to the request. The image analysis task may include a storage address of the target image data in the image management platform. The transmission module may be configured to transmit the image analysis task to the image analysis service platform to direct the image analysis service platform to acquire the target image data from the storage address and perform image analysis on the target image data.

In still another aspect of the present disclosure, a non-transitory computer readable medium is provided. The non-transitory computer readable medium may be included in a gateway device between an image management platform and an image analysis service platform. The non-transitory computer readable medium may include executable instructions that, when executed by at least one processor, direct the at least one processor to perform a method. The method may include obtaining a request for analyzing target image data stored in the image management platform. The method may include generating an image analysis task with respect to the target image data in response to the request. The image analysis task may include a storage address of the target image data in the image management platform. The method may further include transmitting the image analysis task to the image analysis service platform to direct the image analysis service platform to acquire the target image data from the storage address and perform image analysis on the target image data.

In still another aspect of the present disclosure, a method for image analysis is provided. The method may be implemented by an image analysis service platform. The method may include obtaining an image analysis task with respect to target image data. The image analysis task may include a storage address of the target image data in an image management platform. The method may include acquiring the target image data from the storage address in response to the image analysis task. The method may further include performing image analysis on the target image data.

In some embodiments, the image analysis task with respect to the target image data may be transmitted from a gateway device between the image management platform and the image analysis service platform.

In some embodiments, the method may further include generating an analysis abstract of an image analysis result of the target image data; and transmitting the analysis abstract to a gateway device.

In some embodiments, the method may further include obtaining, from a user terminal or the gateway device, a request for obtaining an image analysis result of the target image data; and transmitting the image analysis result to the user terminal.

In still another aspect of the present disclosure, a system for image analysis is provided. The system may include an image analysis service platform. The system may include at least one storage device including a set of instructions; and at least one processor configured to communicate with the at least one storage device. When executing the set of instructions, the at least one processor may be configured to direct the system to perform operations. The operations may include obtaining an image analysis task with respect to target image data. The image analysis task may include a storage address of the target image data in an image management platform. The operations may include acquiring the target image data from the storage address in response to the image analysis task. The operations may further include performing image analysis on the target image data.

In still another aspect of the present disclosure, an image analysis service platform is provided. The image analysis service platform may include an obtaining module and an analysis module. The obtaining module may be configured to obtain an image analysis task with respect to target image data. The image analysis task may include a storage address of the target image data in an image management platform. The obtaining module may be further configured to acquire the target image data from the storage address in response to the image analysis task. The analysis module may be configured to perform image analysis on the target image data.

In still another aspect of the present disclosure, a non-transitory computer readable medium is provided. The non-transitory computer readable medium may be included in a gateway device between an image management platform and an image analysis service platform. The non-transitory computer readable medium may include executable instructions that, when executed by at least one processor, direct the at least one processor to perform a method. The method may include obtaining an image analysis task with respect to target image data. The image analysis task may include a storage address of the target image data in an image management platform. The method may include acquiring the target image data from the storage address in response to the image analysis task. The method may further include performing image analysis on the target image data.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary image analysis system according to some embodiments of the present disclosure;

FIG. 2 is a block diagram illustrating an exemplary gateway device according to some embodiments of the present disclosure;

FIG. 3 is a flowchart illustrating an exemplary process for image analysis according to some embodiments of the present disclosure;

FIG. 4 is a flowchart illustrating an exemplary process for generating an image analysis task according to some embodiments of the present disclosure;

FIG. 5 is a flowchart illustrating an exemplary process for generating an image analysis task according to some embodiments of the present disclosure;

FIG. 6 is a flowchart illustrating an exemplary process 600 for generating an image analysis task according to some embodiments of the present disclosure;

FIG. 7 is a schematic diagram illustrating a structure of an exemplary gateway device according to some embodiments of the present disclosure;

FIG. 8 is a block diagram illustrating an exemplary image analysis service platform according to some embodiments of the present disclosure;

FIG. 9 is a flowchart illustrating an exemplary process for image analysis according to some embodiments of the present disclosure;

FIG. 10 is a flowchart illustrating an exemplary process for image analysis according to some embodiments of the present disclosure; and

FIG. 11 is a schematic diagram illustrating an exemplary computing device according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

Sometimes, an image management platform may send image data to a third-party image analysis service platform (e.g., an AI analysis service platform) for performing image analysis on the image data. At present, the imaging management platform needs to transmit the received image data to the AI image analysis service platform and direct the AI analysis service platform to perform image analysis. However, problems exist in the existing techniques, such as, how to ensure that the image data transmitted to the AI analysis service platform is authorized, how to ensure that the transmitted image data can be processed by the AI algorithm, how to ensure that the transmitted image data is not redundant, how to ensure that the AI analysis results are completely preserved and accurately interpreted and presented, how to quickly transmit the AI analysis results to the user without redundant data, how to reduce changes to the image management platform when adding or updating the Al analysis service platform, etc.

In order to solve the above problems, the present disclosure provides systems and methods for image analysis. The methods may be implemented by a gateway device between an image management platform and an image analysis service platform. The methods may include obtaining a request for analyzing target image data stored in the image management platform. The methods may include generating an image analysis task with respect to the target image data in response to the request. The image analysis task may include a storage address of the target image data in the image management platform. Further, the methods may include transmitting the image analysis task to the image analysis service platform to direct the image analysis service platform to acquire the target image data from the storage address and perform image analysis on the target image data.

According to some embodiments of the present disclosure, by introducing the gateway device, only the image analysis task needs to be transmitted to the image analysis service platform, which can avoid a strong coupling between the image management platform and the image analysis service platform. By transmitting the image analysis task to the image analysis service platform, the image analysis service platform may acquire only the target image data, which may prevent the image analysis service platform from obtaining other image data in the image management platform, thereby improving the privacy and security of image data stored in the image management platform. In addition, only the image analysis task and the target image data need to be transmitted to the image analysis service platform, which can avoid the transmission of redundant data, thereby improving the efficiency of the image analysis and reducing the load of the image analysis platform. Moreover, by introducing the gateway device, a plurality of analyzing devices and algorithms can be added to the image analysis platform, which can expand the capabilities of the image analysis platform without changing the existing structure of the image analysis platform.

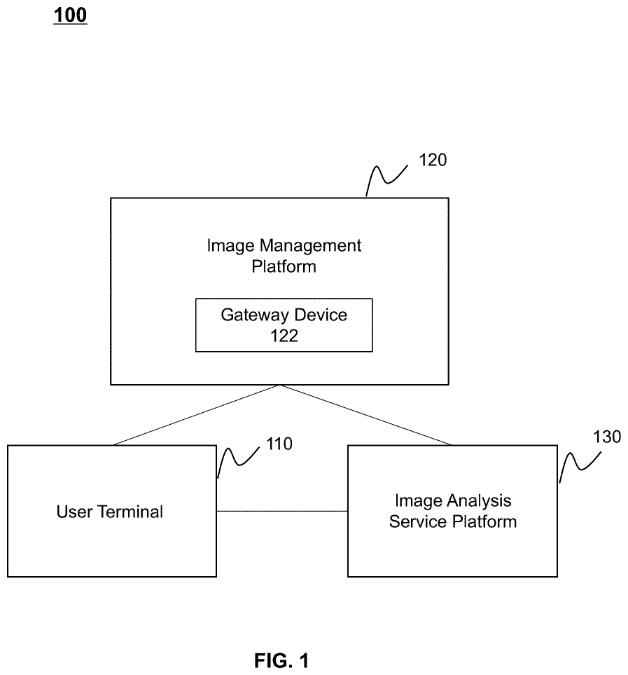

FIG. 1 is a schematic diagram illustrating an exemplary image analysis system 100 according to some embodiments of the present disclosure.

In some embodiments, the image analysis system 100 may be configured to manage and/or analyze image data stored in the image analysis system 100 and/or image data retrieved by the image analysis system 100. The image data may include medical image data (e.g., scanning data, medical image(s), etc.) of subject(s), monitoring images, or the like, or any combination thereof. It should be noted that the description of the image analysis system is merely for illustration, and is not intended to limit the scope of the present disclosure. For example, the image analysis system 100 may be configured to manage and/or analyze text data, voice data, video data, etc.

In some embodiments, the image data may be acquired by an imaging device. For example, an imaging device may include a medical imaging device caused to generate or provide image data by scanning a subject or at least a part of the subject. In some embodiments, the imaging device may include a single modality imaging device. For example, the imaging device may include a positron emission tomography (PET) device, a single-photon emission computed tomography (SPECT) device, a computed tomography (CT) device, a magnetic resonance imaging (MRI) device, a digital subtraction angiography (DSA) system, an intravascular ultrasound (IVUS) device, etc. In some embodiments, the imaging device may include a multi-modality imaging device. Exemplary multi-modality imaging devices may include a positron emission tomography-computed tomography (PET-CT) device, a positron emission tomography-magnetic resonance imaging (PET-MRI) device, a single-photon emission computed tomography-computed tomography (SPECT-CT) device, a digital subtraction angiography-computed tomography (DSA-CT) device, a digital subtraction angiography-positron emission tomography (DSA-PET) device, a digital subtraction angiography-magnetic resonance imaging (DSA-MRI) device, a single photon emission computed tomography-magnetic resonance imaging (SPECT-MRI) device, etc. The multi-modality imaging device may perform multi-modality imaging simultaneously or in sequence.

In some embodiments, the image data may be previously generated and stored in the image analysis system 100 (e.g., a storage device of the image analysis system 100). Alternatively, the image data may be previously generated and stored in an external storage device (e.g., a storage device of the imaging device). The image analysis system 100 may retrieve the image data from the external storage device.

As shown in FIG. 1, the image analysis system 100 may include a user terminal 110, an image management platform 120, and an image analysis service platform 130. In some embodiments, the user terminal 110, the image management platform 120, and/or the image analysis service platform 130 may be connected to and/or communicate with each other via a wireless connection (e.g., a network), a wired connection, or a combination thereof.

The user terminal 110 may provide a user interface via which a user may view information and/or input data and/or instruction(s) (e.g., a request) to the image analysis system 100. For example, a user may send a request to other components (e.g., the image management platform 120 and/or the image analysis service platform 130) through the user terminal 110. For instance, a user may send a request for analyzing target image data stored in the image management platform 120 to the image analysis system 100 (e.g., the image management platform 120) through the user terminal 110. As another example, the user terminal 110 may be used to receive an image analysis result of the target image data from the image analysis service platform 130 and/or display the image analysis result. The user terminal 110 may include a mobile device, a personal computer, a tablet computer, a laptop computer, an Internet of Things (IOT) device, or the like, or any combination thereof.

The image management platform 120 may be configured to manage the image data. For example, the image management platform 120 may be used to store the image data. In some embodiments, the image management platform 120 include a gateway device 122. The gateway device 122 may be used to realize a communication connection (e.g., data transmission) between the image management platform 120 and the image analysis service platform 130. For example, the gateway device 122 may receive the request for analyzing the target image data stored in the image management platform 120 from the user terminal 110 and/or the image management platform 120. In response to the request, the gateway device 122 may generate an image analysis task with respect to the target image data, and transmit the image analysis task to the image analysis service platform 130. The image analysis task may include a storage address of the target image data in the image management platform 120.

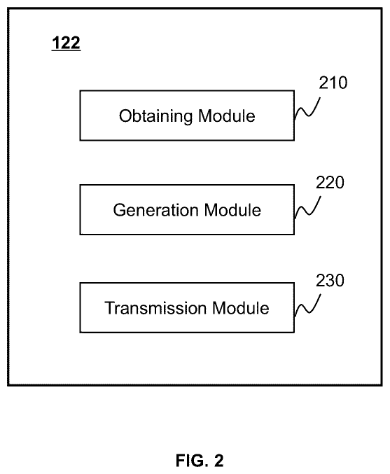

In some embodiments, the gateway device 122 may be integrated into the image management platform 120 as shown in FIG. 1. For example, the gateway device 122 may be a processor, a server, or a server cluster (which includes multiple micro servers) in the image management platform 120. In some embodiments, the gateway device 122 may be device independent from the image management platform 120. More descriptions of the gateway device may be found elsewhere in the present disclosure (e.g., FIGS. 2-7 and the descriptions thereof).

In some embodiments, the image management platform 120 may include at least one first storage device (not shown). In some embodiments, the at least one first storage device may include a mass storage, a removable storage, a volatile read-and-write memory, a read-only memory (ROM), or the like, or any combination thereof. In some embodiments, the at least one first storage device may store the image data.

The image analysis service platform 130 may provide image analysis service. For example, the image analysis service platform 130 may be configured to acquire the target image data from the storage address and perform image analysis on the target image data. In some embodiments, the image analysis service platform 130 may perform image analysis using one or more of a plurality of candidate algorithms (e.g., a plurality of trained image analysis models). In some embodiments, the image analysis service platform 130 may include a plurality of analyzing devices (e.g., processors, servers) each of which is used to execute one or more specific candidate algorithms. The process of selecting a target algorithm for processing the target image data may also be regarded as a process of selecting a target analyzing device that can implement the target algorithm.

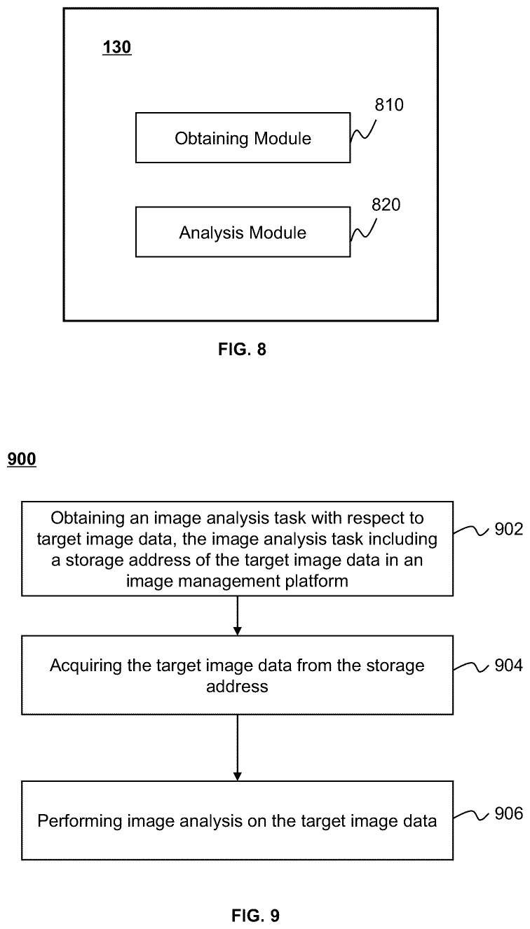

In some embodiments, the image analysis service platform 130 may include at least one second storage device (not shown). In some embodiments, the at least one second storage device may include a mass storage, a removable storage, a volatile read-and-write memory, a read-only memory (ROM), or the like, or any combination thereof. In some embodiments, the at least one second storage device may store the plurality of candidate algorithms and analysis results. More descriptions the image analysis service platform may be found elsewhere in the present disclosure (e.g., FIGS. 8-9 and the descriptions thereof).

In some embodiments, the image management platform 120 and/or the image analysis service platform 130 may be a single server or a server group. The server group may be centralized or distributed. In some embodiments, the image management platform 120 and/or the image analysis service platform 130 may be local or remote. In some embodiments, the image management platform 120 and/or the image analysis service platform 130 may be implemented on a cloud platform.

In some embodiments, the image management platform 120 and/or the image analysis service platform 130 may be implemented by a computing device. For example, the computing device may include a processor, a storage, an input/output (I/O), and a communication port. The processor may execute computer instructions (e.g., program codes) and perform functions of the image management platform 120 and/or the image analysis service platform 130 in accordance with the techniques described herein. The computer instructions may include, for example, routines, programs, objects, components, data structures, procedures, modules, and functions, which perform particular functions described herein. In some embodiments, the image management platform 120 and/or the image analysis service platform 130, or a portion of the image management platform 120 and/or the image analysis service platform 130 may be implemented by a portion of the user terminal 110.

In some embodiments, the image management platform 120 and/or the image analysis service platform 130 may include multiple processing devices. Thus, operations and/or method steps that are performed by one processing device as described in the present disclosure may also be jointly or separately performed by the multiple processing devices. For example, if in the present disclosure, the image analysis system 100 executes both operation A and operation B, it should be understood that operation A and operation B may also be performed by two or more different processing devices jointly or separately (e.g., a first processing device executes operation A and a second processing device executes operation B, or the first and second processing devices jointly execute operations A and B).

It should be noted that the image analysis system 100 is provided for illustration purposes, and is not intended to limit the scope of the present disclosure. For persons having ordinary skills in the art, multiple variations and modifications may be made under the teachings of the present disclosure. However, those variations and modifications do not depart from the scope of the present disclosure. For example, the image analysis system 100 may include one or more additional components and/or one or more components of the image analysis system 100 described above may be omitted. Additionally or alternatively, two or more components of the image analysis system 100 may be integrated into a single component. A component of the image analysis system 100 may be implemented on two or more sub-components.

FIG. 2 is a block diagram illustrating an exemplary gateway device 122 according to some embodiments of the present disclosure. In some embodiments, the gateway device 122 may be in communication with a computer-readable storage medium and the modules of the gateway device 122 may execute instructions stored in the computer-readable storage medium.

As illustrated in FIG. 2, the gateway device 122 may include an obtaining module 210, a generation module 220, and a transmission module 230.

The obtaining module 210 may be configured to obtain, from an image management platform, a request for analyzing target image data stored in the image management platform. The target image data may refer to image data that needs to be analyzed. The request for analyzing the target image data may include information relating to the target subject, information relating to the target image data, analysis information, or the like, or any combination thereof. More descriptions regarding the obtaining of the request for analyzing target image data may be found elsewhere in the present disclosure. See, e.g., operation 302 and relevant descriptions thereof.

The generation module 220 may be configured to generate an image analysis task with respect to the target image data. The image analysis task may include a storage address of the target image data in the image management platform. More descriptions regarding the generation of the image analysis task may be found elsewhere in the present disclosure. See, e.g., operation 304 and relevant descriptions thereof.

The transmission module 230 may be configured to transmit the image analysis task to the image analysis service platform to direct the image analysis service platform to acquire the target image data from the storage address and perform image analysis on the target image data. More descriptions regarding the transmission of the image analysis task may be found elsewhere in the present disclosure. See, e.g., operation 306 and relevant descriptions thereof.

The obtaining module 210 may be further configured to obtain an image analysis report of the target image data. The image analysis report may include an analysis abstract, an analysis status, an image analysis result, or the like, or any combination thereof. More descriptions regarding the obtaining of the image analysis report may be found elsewhere in the present disclosure. See, e.g., operation 308 and relevant descriptions thereof.

It should be noted that the above descriptions of the gateway device 122 are provided for the purposes of illustration, and are not intended to limit the scope of the present disclosure. For persons having ordinary skills in the art, various variations and modifications may be conducted under the guidance of the present disclosure. However, those variations and modifications do not depart from the scope of the present disclosure. In some embodiments, the gateway device 122 may include one or more other modules. For example, the gateway device 122 may include a storage module to store data generated by the modules in the gateway device 122. In some embodiments, any two of the modules may be combined as a single module, and any one of the modules may be divided into two or more units.

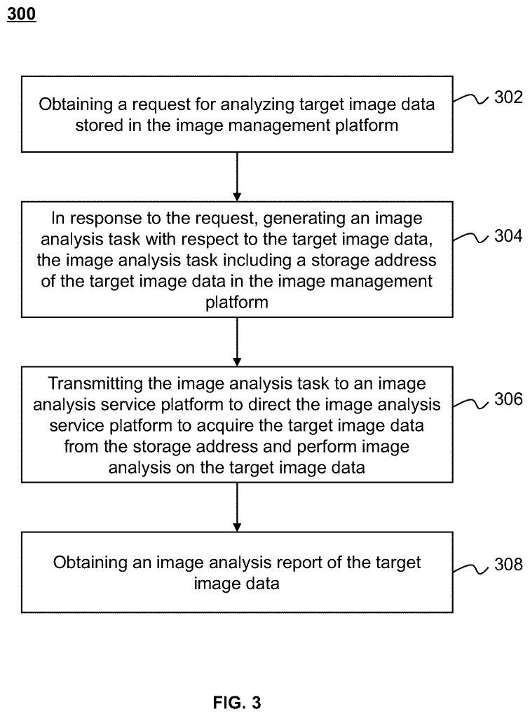

FIG. 3 is a flowchart illustrating an exemplary process 300 for image analysis according to some embodiments of the present disclosure. In some embodiments, the process 300 may be implemented in the gateway device 122 illustrated in FIG. 2. For example, the process 300 may be stored in a storage device (e.g., a storage device of the gateway device 122, an external storage device) in the form of instructions (e.g., an application), and invoked and/or executed by the gateway device 122 (e.g., a processor of the gateway device 122).

In 302, the gateway device 122 (e.g., the obtaining module 210) may obtain a request for analyzing target image data stored in the image management platform.

The target image data may refer to image data that needs to be analyzed. For example, the target image data may include scanning data, medical image(s), etc., of a target subject that needs to be diagnosed and/or treated.

The request for analyzing the target image data may include information relating to the target subject, information relating to the target image data, analysis information, or the like, or any combination thereof. The information relating to the target subject may include an identification, a gender, a name, an age, a region of interest (ROI), etc., of the target subject. The information relating to the target image data may include an identification, a type, etc., of the target image data. The analysis information may include an analysis manner, an analysis requirement (e.g., a required time), etc.

In some embodiments, the gateway device 122 may obtain the request from the image management platform. For example, a user (e.g., a requester, a doctor, a technician, etc.) may send the request to the image management platform 120 through the user terminal 110, and the gateway device 122 may obtain the request from the image management platform 120. Merely by way of example, a requester may select a request initiation option via an image analysis interface of the user terminal 110, and then the image analysis interface may be switched to a selection interface of subjects and/or image data. The requester may determine the target subject and/or the target image data from the subjects and/or the image data via the selection interface. That is, the requester may authorize the image analysis on the target subject and/or the target image data. Accordingly, the request for analyzing the target image data may be sent to the image management platform 120, and then the gateway device 122 may obtain the request from the image management platform 120.

In some embodiments, the gateway device 122 may directly obtain the request from the user terminal 110. For example, a requester may send the request to the gateway device 122 of the image management platform 120 through the user terminal 110.

In 304, in response to the request, the gateway device 122 (e.g., the generation module 220) may generate an image analysis task with respect to the target image data. The image analysis task may include a storage address of the target image data in the image management platform.

The image analysis task may be used to direct an image analysis service platform to analyze the target image data and include rules or requirements specifying how to analyze the target image data. The image analysis task may include a task type, an analysis algorithm, an identification of a target analyzing device, the storage address, a task sending mode, or the like, or any combination thereof. As used herein, the task type may include processing the target image data and/or analyzing the target image data. The analysis algorithm (also referred to as a target algorithm) may be used to process the target image data. The target analyzing device may be used to perform the image analysis. For example, the target analyzing device may correspond to the analysis algorithm, and perform the analysis algorithm to process the target image data.

In some embodiments, the gateway device 122 may determine whether the image analysis task needs to be generated. For example, the gateway device 122 may obtain one or more features of the target image data based on the request for analyzing the target image data, and determine whether the one or more features of the target image data satisfy a first condition. The first condition may be used to determine the availability of the target image data, for example, whether the target image data can be processed by the image analysis service platform. If the one or more features of the target image data satisfy the first condition, the gateway device 122 may determine that the image analysis task needs to be generated, and generate the image analysis task with respect to the target image data.

As another example, the gateway device 122 may obtain user permission corresponding to the request for analyzing the target image data, and determine whether the user permission corresponding to the request satisfies a second condition. If the user permission corresponding to the request satisfies the second condition, the gateway device 122 may determine that the image analysis task needs to be generated, and generate the image analysis task with respect to the target image data.

As still another example, the gateway device 122 may determine whether image analysis has been performed on the target image data. If image analysis has not been performed on the target image data (i.e., the target image data has been analyzed), the gateway device 122 may determine that the image analysis task needs to be generated, and generate the image analysis task with respect to the target image data.

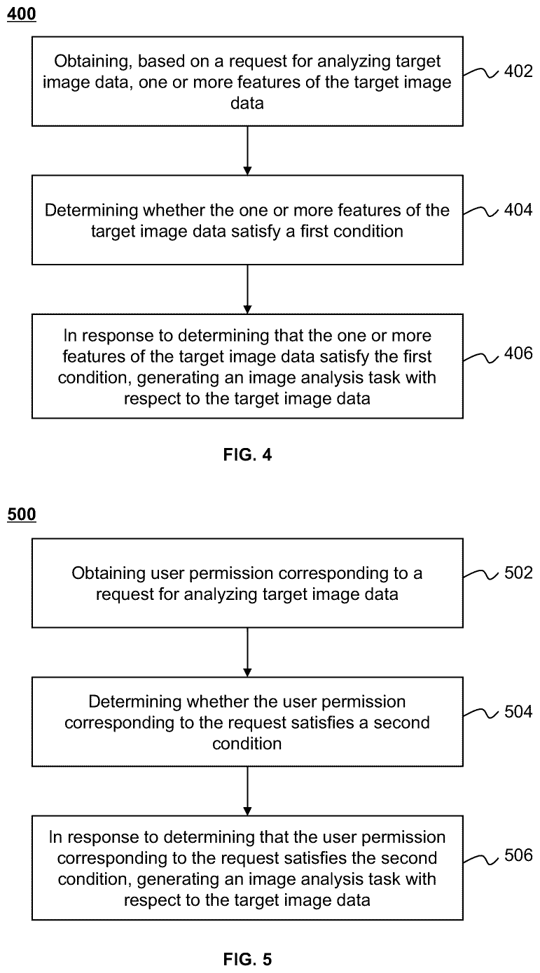

In some embodiments, the gateway device 122 may generate the image analysis task with respect to the target image data based on the analysis algorithm. For example, the gateway device 122 may obtain a plurality of candidate algorithms, and determine a target algorithm (i.e., the analysis algorithm) from the plurality of candidate algorithms based on the target image data. Further, the gateway device 122 may generate the image analysis task with respect to the target image data based on the target algorithm. More descriptions regarding the generation of the image analysis task may be found elsewhere in the present disclosure (e.g., FIGS. 4-7 and the descriptions thereof).

In some embodiments, the gateway device 122 may generate the image analysis task with respect to the target image data based on a custom rule. The custom rule may relate to preference, requirements, and/or habits of the user (e.g., the requestor). In some embodiments, the custom rule may be set by a user or determined by a common rule engine (e.g., a dependent processing device, part of the gateway device 122 or the image management platform). For example, when a custom rule is added or modified, the gateway device 122 may modify configuration file(s) corresponding to the added or modified custom rule using the common rule engine without changing a data processing process performed by the gateway device 122.

In some embodiments, the gateway device 122 may include a task constructor. When the image analysis task needs to be generated, the gateway device 122 may generate the image analysis task using the task constructor. For example, the gateway device 122 may use the task constructor to generate the image analysis task based on the analysis algorithm, the storage address of the target image data in the image management platform, etc.

In 306, the gateway device 122 (e.g., the transmission module 230) may transmit the image analysis task to the image analysis service platform to direct the image analysis service platform to acquire the target image data from the storage address and perform image analysis on the target image data.

In some embodiments, when the image analysis task is generated, the gateway device 122 may transmit the image analysis task to the image analysis service platform. For example, once the image analysis task is generated, the gateway device 122 may transmit the image analysis task to the image analysis service platform immediately. As another example, the gateway device 122 may transmit the image analysis task to the image analysis service platform at a certain time. The certain time may be determined based on actual conditions (e.g., an idle time of the image analysis service platform, a time when an image analysis result is required, etc.).

In some embodiments, the gateway device 122 may include a task database, and the gateway device 122 may store the image analysis task in the task database. For example, the task database may be a general relational database, such as, a Hadoop database (HBase), a Cassandra database, etc. In some embodiments, a plurality of image analysis tasks may be stored in the task database. The gateway device 122 may transmit the image analysis task to the image analysis service platform based on a task queue of the plurality of image analysis tasks in the task database. The task queue may be a general message queue or a database table. In some embodiments, the task queue may be established based on a comprehensive evaluation on the plurality of image analysis tasks in the task database. For example, the gateway device 122 may generate a task queue by comprehensively evaluating the timeliness requirement, the importance degree, the priority degree, the task initiation time, etc., of each of the plurality of image analysis tasks in the task database, and transmit the image analysis task to the image analysis service platform based on the task queue. For example, the image analysis tasks in the task queue may be transmitted to the image analysis service platform in sequence.

In some embodiments, the gateway device 122 may determine whether the image analysis service platform is available. If the image analysis service platform is available, the gateway device 122 may transmit the image analysis task to the image analysis service platform.

Merely by way of example, the gateway device 122 may determine whether a target analyzing device corresponding to the target algorithm has enough resources to run the image analysis task. It should be noted that a plurality of image analysis tasks may be performed in the target analyzing device simultaneously or sequentially. If the target analyzing device performs the image analysis task together with other image analysis tasks simultaneously, the gateway device 122 should determine whether the remaining resource of the target analyzing device is enough to run the image analysis task. For example, the gateway device 122 may determine a running status and a remaining resource of the target analyzing device corresponding to the target algorithm by using a processing status reader and a status data parser to process information of the target analyzing device. As another example, the gateway device 122 may determine a running status and a remaining resource of the target analyzing device corresponding to the target algorithm by querying the target analyzing device through a preset query interface. Further, the gateway device 122 may store the running status and the remaining resource of the target analyzing device in a storage device (e.g., the task database) of the gateway device 122 for determining whether the image analysis service platform is available. If the remaining resource of the target analyzing device is enough to run the image analysis task, the gateway device 122 may transmit the image analysis task to the image analysis service platform 130 (e.g., the target analyzing device).

By evaluating the remaining resource of the target analyzing device corresponding to the target algorithm, the image analysis task may be processed when the target analyzing device is available, which can improve the efficiency of image analysis.

In some embodiments, after the image analysis task is transmitted to the image analysis service platform, the gateway device 122 may direct the image analysis service platform to acquire the target image data from the storage address. Therefore, only the target image data can be acquired by the image analysis service platform, which may prevent other image data from being transmitted to the image analysis service platform, thereby improving the privacy and security of the other image data.

In some embodiments, after the target image data is acquired by the image analysis service platform, the image analysis service platform may perform the image analysis on the target image data. For example, the image analysis service platform may process the target image data using the target algorithm. If the image analysis service platform includes multiple analyzing devices, the image analysis service platform may allocate the image analysis task to the target analyzing device corresponding to the target algorithm.

In 308, the gateway device 122 (e.g., the obtaining module 210) may obtain an image analysis report of the target image data.

In some embodiments, after the image analysis is performed on the target image data, the gateway device 122 may obtain, from the image analysis service platform, the image analysis report of the target image data. The image analysis report may include an analysis abstract, an analysis status, an image analysis result, or the like, or any combination thereof.

The analysis abstract may include a summary of the image analysis result and/or important information in the image analysis result. For example, the analysis abstract may include diagnosis information (e.g., a negative result or a positive result) of an ROI (e.g., lesion), prompt information of the ROI, an obtaining manner of the image analysis result, etc. In some embodiments, the gateway device 122 may obtain the analysis abstract of the target image data from the image analysis service platform, and manage the analysis abstract. For example, the gateway device 122 may store the analysis abstract in a storage component of the gateway device 122. For instance, the gateway device 122 may persistently store the analysis abstract in the task database of the gateway device 122.

In some embodiments, the gateway device 122 may transmit the analysis abstract to a user terminal (e.g., the user terminal 110). For instance, the gateway device 122 may determine whether the image analysis task corresponding to the target subject is stored in a task database based on the information of the target subject. If the image analysis task corresponding to the target subject is stored in the task database, the gateway device 122 may send an analysis abstract of the target subject to the user terminal 110. As another example, the gateway device 122 may direct the user terminal 110 to display the analysis abstract of the target subject. As yet another example, the gateway device 122 may transmit the analysis abstract to the user terminal if the analysis abstract satisfies a condition (e.g., includes a positive result of lesion).

By obtaining the analysis abstract, key information (e.g., the diagnosis information, the prompt information, etc.) relating to the target image data may be provided to the user (e.g., the requester), which can reduce time spent on irrelevant details, thereby improving the experience of the user and the efficiency of the image analysis.

The analysis status may indicate an analysis progress of the image analysis. For example, if the image analysis task is transmitted to the image analysis service platform, the gateway device 122 may transmit a status query request to the image analysis service platform at a preset time, and receive the analysis status from the image analysis service platform. As another example, when the image analysis task has been performed, the gateway device 122 may receive an analysis status indicating that the image analysis task has been performed. By obtaining the analysis status, the availability of the image analysis service platform and the analysis progress of the image analysis may be determined. Therefore, the resources of the image analysis service platform (e.g., the target analyzing device) can be allocated reasonably after the image analysis task is completed.

The image analysis result may refer to a detailed analysis report of the target image data. For example, the image analysis result may include diagnosis information and conclusions of the image analysis in the form of lists, images, symbolic marks, etc. In some embodiments, since the image analysis result includes more information and requires more storage spaces than the analysis abstract, the image analysis result may be stored in the image analysis service platform instead of the gateway device 122.

The gateway device 122 may obtain a second request for obtaining the image analysis result of the target image data from the user terminal, and transmit the second request to the image analysis service platform to direct the image analysis service platform to transmit the image analysis result to the user terminal.

In some embodiments, the image analysis task may include a storage time period. After the image analysis task is performed and the storage time period is reached, the gateway device 122 may direct the image analysis service platform to delete the target image data. By deleting the target image data, information leakage due to a permanent storage of the target image data on a third-party platform (e.g., the image analysis service platform 130) may be avoided, which can improve the privacy and security of the target image data.

According to some embodiments of the present disclosure, by introducing the gateway device 122, only the image analysis task needs to be transmitted to the image analysis service platform, which can avoid a strong coupling between the image management platform and the image analysis service platform. By transmitting the image analysis task to the image analysis service platform, the image analysis service platform may acquire only the target image data, which may prevent the image analysis service platform from obtaining other image data in the image management platform, thereby improving the privacy and security of the image data. In addition, only the image analysis task and the target image data need to be transmitted to the image analysis service platform, which can avoid the transmission of redundant data, thereby improving the efficiency of the image analysis and reducing the load of the image analysis platform. Moreover, by introducing the gateway device, a plurality of analyzing devices and algorithms can be added to the image analysis platform, which can expand the capabilities of the image analysis platform without changing the existing data storage of the image analysis platform.

FIG. 4 is a flowchart illustrating an exemplary process 400 for generating an image analysis task according to some embodiments of the present disclosure. In some embodiments, the process 400 may be performed to achieve at least part of operation 304 as described in connection with FIG. 3.

In 402, the gateway device 122 (e.g., the generation module 220) may obtain, based on a request for analyzing target image data, one or more features of the target image data.

The one or more features of the target image data may include metadata information of the target image data. As used herein, the metadata information may include information regarding an examination type, an examination part, a data owner, a privacy level, information of the target subject, institution information, an image storage address, an image quantity, an image quality, an image format, an image acquisition manner, an image acquisition time, or the like, or any combination thereof. The image quality may include an image resolution, etc. The image format may include DICOM, nii, etc. The image acquisition manner may include the type of device for obtaining the target image data, such as a CT device, an MR device, a PET device, etc.

In some embodiments, the request for analyzing the target image data may include information relating to the target subject, such as, an identification of the target subject. The gateway device 122 may obtain the one or more features of the target image data based on the information relating to the target subject (e.g., the identification of the target subject). For example, after the identification of the target subject is obtained, the gateway device 122 may obtain the target image data in the image management platform 120, and determine the one or more features of the target image data based on the target image data. As another example, when the target image data is stored in the image management platform 120, the one or more features of the target image data may be input and/or stored in the image management platform 120. Therefore, the gateway device 122 may retrieve the one or more features of the target image data from the image management platform 120 based on the identification of the target subject.

In 404, the gateway device 122 (e.g., the generation module 220) may determine whether the one or more features of the target image data satisfy a first condition.

The first condition may be used to determine the availability of the target image data, for example, whether the target image data can be processed by the image analysis service platform (e.g., using AI algorithms). In some embodiments, the first condition may relate to the one or more features. For example, the first condition may be determined by manually or automatically setting one or more threshold values (or reference values) for the one or more features. As another example, the first condition may be determined based on analysis algorithms or analyzing devices in the image analysis service platform 130.

In some embodiments, the gateway device 122 may compare the one or more features of the target image data with their corresponding threshold values (or reference values) in the first condition to determine whether the one or more features of the target image data satisfy the first condition. For example, the gateway device 122 may compare the image quantity, the image quality, the image storage address, the image acquisition manner, etc., of the target image data with their corresponding threshold values (or reference values) in the first condition to determine whether the one or more features of the target image data satisfy the first condition.

Merely by way of example, the first condition may include that a preset examination part is the heart, a preset image format is the DICOM format, and a preset image acquisition time is a certain time period (e.g., Monday). The gateway device 122 may compare an examination part, an image format, and an image acquisition time of the target image data with the preset examination part, the preset image format, and the preset image acquisition time. That is, the gateway device 122 may determine whether the examination part of the target image data is consistent with the preset examination part (e.g., the heart), whether the image format of the target image data is consistent with the preset image format (e.g., the DICOM format), and the image acquisition time of the target image data is consistent with the preset image acquisition time (e.g., Monday). If the examination part of the target image data is the heart, the image format of the target image data is the DICOM format, and the image acquisition time of the target image data is Monday, the gateway device 122 may determine that the one or more features of the target image data satisfy the first condition, and the process 400 may proceed to operation 406. If the examination part of the target image data is not the heart, or the image format of the target image data is not the DICOM format, or the image acquisition time of the target image data is not Monday, the gateway device 122 may determine that the one or more features of the target image data do not satisfy the first condition. The gateway device 122 may send the determination result that the one or more features of the target image data do not satisfy the first condition to the user terminal 110.

In 406, in response to determining that the one or more features of the target image data satisfy the first condition, the gateway device 122 (e.g., the generation module 220) may generate an image analysis task with respect to the target image data.

For example, the gateway device 122 may generate the image analysis task with respect to the target image data based on the request for analyzing the target image data.

In some embodiments, the gateway device 122 may further determine whether the image analysis task needs to be generated. For example, the gateway device 122 may determine whether user permission corresponding to the request satisfies a second condition. As another example, the gateway device 122 may determine whether the image analysis has been performed on the target image data.

In some embodiments, the gateway device 122 may generate the image analysis task with respect to the target image data based on an analysis algorithm. More descriptions regarding the generation of the image analysis task may be found elsewhere in the present disclosure (e.g., FIGS. 3, 5, and 6, and the descriptions thereof).

According to some embodiments of the present disclosure, by determining whether the one or more features of the target image data satisfy the first condition, the availability of the target image data may be determined, which can improve the accuracy and efficiency of the generation of the image analysis task, thereby improving the accuracy and efficiency of the image analysis.

FIG. 5 is a flowchart illustrating an exemplary process 500 for generating an image analysis task according to some embodiments of the present disclosure. In some embodiments, the process 500 may be performed to achieve at least part of operation 304 as described in connection with FIG. 3.

In 502, the gateway device 122 (e.g., the generation module 220) may obtain user permission corresponding to a request for analyzing target image data.

The user permission may include a first user permission of a user (e.g., a requester) who initiates the request and a second user permission of the target image data and/or a target subject. As used herein, the first user permission may include an authority level of the user, and the second user permission may include a privacy level of the target image data, a privacy level of the target subject, a privacy level of a data owner of the target image data, a privacy level of the institution who owns the target image data, etc.

In some embodiments, when the user interacts with the gateway device 122 (or the image analysis system 100) via the user terminal, the user may need to login, and the gateway device 122 may obtain the first user permission based on login information of the user.

In some embodiments, the request for analyzing target image data may include information relating to the target subject, such as, an identification of the target subject. The gateway device 122 may obtain the second user permission based on the information relating to the target subject (e.g., the identification of the target subject). For example, when the target image data is stored in the image management platform 120, the second user permission may be input and/or stored in the image management platform 120 or a user database connected to the image management platform 120. Therefore, the gateway device 122 may retrieve the second user permission from the image management platform 120 or the user database based on the identification of the target subject.

In 504, the gateway device 122 (e.g., the generation module 220) may determine whether the user permission corresponding to the request satisfies a second condition.

The second condition may include a corresponding relationship between the first user permission and the second user permission. For example, the corresponding relationship may indicate that a level of the first user permission needs to be higher than a level of the second user permission.

In some embodiments, the gateway device 122 may compare the level of the first user permission with the level of the second user permission to determine whether the user permission corresponding to the request satisfies the second condition. For example, the gateway device 122 may compare the authority level of the user with the privacy level of the target subject. If the authority level of the user is equal to or higher than the privacy level of the target subject, the gateway device 122 may determine that the user permission corresponding to the request satisfies the second condition, and the process 500 may proceed to operation 506. If the authority level of the user is not higher than the privacy level of the target subject, the gateway device 122 may determine that the user permission corresponding to the request does not satisfy the second condition, and the gateway device 122 may send the determination result that the user permission corresponding to the request does not satisfy the second condition to the user terminal 110.

In 506, in response to determining that the user permission corresponding to the request satisfies the second condition, the gateway device 122 (e.g., the generation module 220) may generate an image analysis task with respect to the target image data.

For example, the gateway device 122 may generate the image analysis task with respect to the target image data based on the request for analyzing the target image data.

In some embodiments, the gateway device 122 may further determine whether the image analysis task needs to be generated. For example, the gateway device 122 may determine whether one or more features of the target image data satisfy a first condition. As another example, the gateway device 122 may determine whether the image analysis has been performed on the target image data.

In some embodiments, the gateway device 122 may generate the image analysis task with respect to the target image data based on an analysis algorithm. More descriptions regarding the generation of the image analysis task may be found elsewhere in the present disclosure (e.g., FIGS. 3, 4, and 6, and the descriptions thereof).

According to some embodiments of the present disclosure, by determining whether the user permission corresponding to the request satisfies the second condition, the user permission corresponding to the request may be verified, which can improve the security of the target image data, thereby improving the accuracy and efficiency of the generation of the image analysis task.

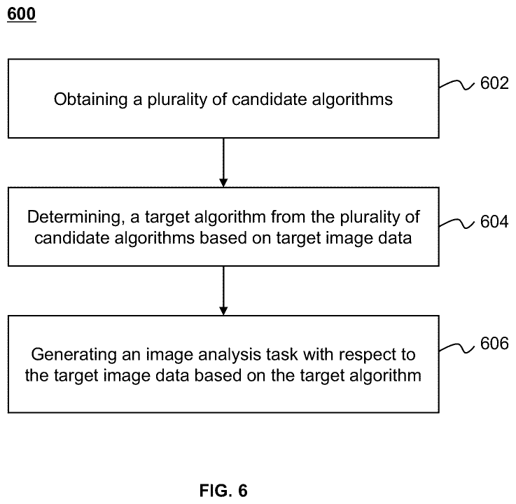

FIG. 6 is a flowchart illustrating an exemplary process 600 for generating an image analysis task according to some embodiments of the present disclosure. In some embodiments, the process 600 may be performed to achieve at least part of operation 304 as described in connection with FIG. 3.

In 602, the gateway device 122 (e.g., the generation module 220) may obtain a plurality of candidate algorithms.

The candidate algorithms refer to image analysis algorithms that the image analysis service platform can implement. Each of the plurality of candidate algorithms may be used to implement specific functions and/or process one or more specific types of image data. In some embodiments, each of the plurality of candidate algorithms may be executed by one analyzing device in an image analysis serve platform (e.g., the image analysis serve platform 130). That is, the gateway device 122 may obtain the plurality of candidate algorithms that can be executed by the plurality of analyzing devices in the image analysis serve platform. For example, the gateway device 122 may obtain a list or a table that is used to store the plurality of candidate algorithms that can be executed by the plurality of analyzing devices in the image analysis serve platform 130.

In some embodiments, the plurality of candidate algorithms may be stored in an algorithm database (e.g., a table, a list, etc.), and the gateway device 122 may obtain the plurality of candidate algorithms from the algorithm database. The algorithm database may include other information of the plurality of candidate algorithms. For example, for each of the plurality of candidate algorithms, the other information may include a storage address, a corresponding analyzing device, a function, an image processing capacity, requirement(s) with respect to an input of the candidate algorithm, etc.

In 604, the gateway device 122 (e.g., the generation module 220) may determine a target algorithm from the plurality of candidate algorithms based on target image data.

The target algorithm may be used to process the target image data.

In some embodiments, for each of the plurality of candidate algorithms, the gateway device 122 may determine at least one mandatory requirement and at least one optional requirement with respect to an input of the candidate algorithm. A mandatory requirement may refer to a requirement that the input of the candidate algorithm must satisfy. For example, for a candidate algorithm that is only used to process CT image data, the mandatory requirement may include a requirement that the input includes CT image data. An optional requirement may refer to a non-mandatory requirement regarding the input of the candidate algorithm. For example, the optional requirement may include a requirement that a noise of the input does not exceed a noise threshold.

Merely by way of example, for each of the plurality of candidate algorithms, the gateway device 122 may obtain the at least one mandatory requirement and the at least one optional requirement with respect to an input of the candidate algorithm from the algorithm database. Then, the gateway device 122 may select at least one candidate algorithm from the plurality of candidate algorithms. The target image data may satisfy the at least one mandatory requirement of each selected candidate algorithm. The gateway device 122 may further determine the target algorithm from the at least one selected candidate algorithm based on the target image data and the at least one optional requirement of each selected candidate algorithm. For example, if the target image data satisfies each optional requirement of one or more specific candidate algorithms, the gateway device 122 may randomly select a candidate algorithm from the one or more specific candidate algorithms as the target algorithm. As another example, the gateway device 122 may determine a score of each of the at least one selected candidate algorithm based on the target image data and the at least one optional requirement of the selected candidate algorithm, and designate a candidate algorithm with a highest score among the at least one selected candidate algorithm as the target algorithm. The score of a selected candidate algorithm may indicate the degree that the target image data satisfies the at least one optional re-equipment of the selected candidate algorithm. The score may be determined based on a scoring rule or a scoring model (e.g., a trained machine learning model). Further, if there are a plurality of candidate algorithms with the highest score, the gateway device 122 may randomly select one candidate algorithm from the plurality of candidate algorithms with the highest score, and designate the candidate algorithm as the target algorithm.

In some embodiments, the gateway device 122 may obtain a corresponding relationship between the plurality of candidate algorithms and a plurality of reference feature vectors representing a plurality of sets of reference image data. The corresponding relationship may be determined based on historical usage records. For example, if a candidate algorithm is often used to process a type of reference feature vectors (or a type of sets of reference image data), it may be considered that there is a correspondence between the candidate algorithm and the type of reference feature vectors (or the type of sets of reference image data). The corresponding relationship may be denoted in a table, a diagram, a mathematic function, etc. The reference feature vector of a set of reference image data may be determined based on an image type, an examination part, features of a reference subject, etc., corresponding to the set of reference image data. The gateway device 122 may determine a target feature vector representing the target image data. The determination of the target feature vector may be similar to the determination of the reference feature vector.

Further, the gateway device 122 may determine the target algorithm from the plurality of candidate algorithms based on the target feature vector and the corresponding relationship. For example, the corresponding relationship may be denoted in a table including a plurality of rows, each of the rows may record a reference feature vector and a corresponding candidate algorithm. The gateway device 122 may retrieve the target algorithm by looking up the table (i.e., the corresponding relationship) based on the target feature vector. For instance, the gateway device 122 may determine one or more reference feature vectors from the plurality of reference feature vectors. A similarity degree between each of the one or more reference feature vectors and the target feature vector may exceed a similarity threshold. The similarity degree may be determined based on a similarity rule or a similarity model (e.g., a trained machine learning model). The similarity threshold may be determined based on a system default setting (e.g., statistic information) or set manually by a user (e.g., a requester, a technician, a doctor, a physicist, etc.), such as, 0.6, 0.7, 0.8, 0.9, etc. The gateway device 122 may determine the target algorithm based on the corresponding relationship and a reference feature vector with a highest similarity degree among the one or more reference feature vectors. For example, the candidate algorithm that corresponds to the reference feature vector with the highest similarity degree may be designated as the target algorithm.

In some embodiments, the gateway device 122 may obtain an algorithm determination model. The gateway device 122 may determine the target algorithm based on the target image data using the algorithm determination model. For example, the gateway device 122 may input the target image data of the target subject into the algorithm determination model, and the algorithm determination model may output the target algorithm. As another example, the gateway device 122 may determine a target feature vector representing the target image data, and input the target feature vector into the algorithm determination model. The algorithm determination model may output the target algorithm.

In some embodiments, the algorithm determination model may refer to a process or an algorithm used for determining a recommended algorithm used for processing image data. The algorithm determination model may be a trained machine learning model. Exemplary machine learning models may include a convolutional neural network (CNN) model, a recurrent neural network (RNN) model, a long short term memory (LSTM) network model, a fully convolutional neural network (FCN) model, a generative adversarial network (GAN) model, or the like, or any combination thereof.

In some embodiments, the gateway device 122 may obtain the algorithm determination model from a storage device of the gateway device 122 or a third-party database. In some embodiments, the algorithm determination model may be generated by the gateway device 122 or another computing device according to a machine learning algorithm. In some embodiments, the algorithm determination model may be generated by a computing device (e.g., the gateway device 122) by training an initial model using a plurality of training samples. Each of the plurality of training samples may include sample image data (or a sample feature vector of the sample image data) of a sample subject and a sample algorithm used to process the sample image data. The sample algorithm may be determined automatically or manually. For example, a user may determine the sample algorithm corresponding to the sample image data (or the sample feature vector) via a user terminal (e.g., the user terminal 110) that displays the sample image data (or the sample feature vector). As another example, the gateway device 122 may determine the sample algorithm corresponding to the sample image data (or the sample feature vector) based on an analyzing record of the sample image data.

In 606, the gateway device 122 (e.g., the generation module 220) may generate the image analysis task with respect to the target image data based on the target algorithm.

For example, the generated image analysis task with respect to the target image data may include information (e.g., an identification) relating to the target algorithm.

In some embodiments, the gateway device 122 may further determine whether the image analysis task needs to be generated by performing the process 400 and/or the process 500. More descriptions the generation of the image analysis task may be found elsewhere in the present disclosure (e.g., FIGS. 3-5 and the descriptions thereof).

According to some embodiments of the present disclosure, the target algorithm may be automatically determined from the plurality of candidate algorithms based on target image data, and the image analysis task with respect to the target image data may be generated based on the target algorithm, which can improve the accuracy and efficiency of image analysis.

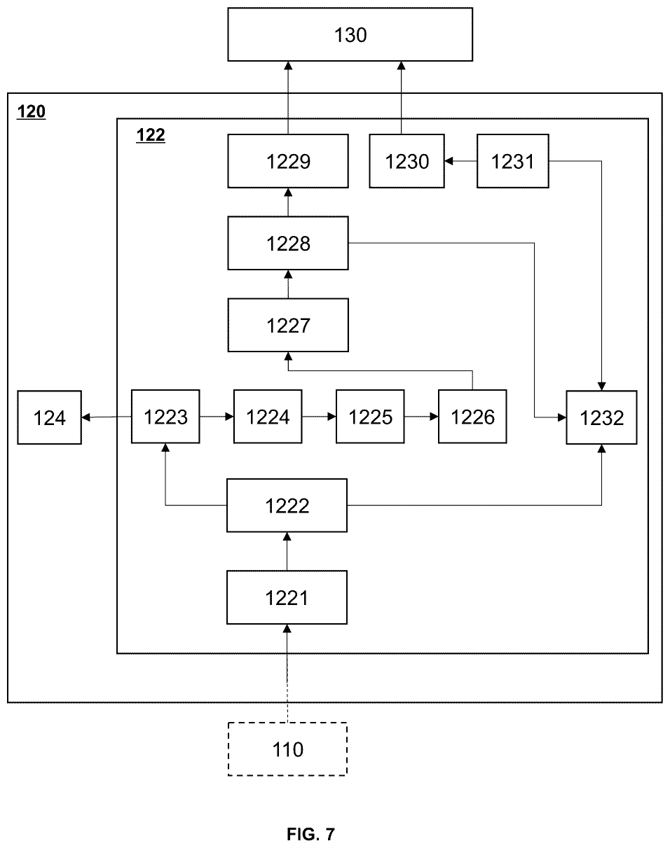

FIG. 7 is a schematic diagram illustrating a structure of an exemplary gateway device 122 according to some embodiments of the present disclosure.

As illustrated in FIG. 7, the gateway device 122 may include a request receiver 1221, a request result retriever 1222, an image data retriever 1223, a data availability checker 1224, a privacy and authorization filter 1225, a custom rule filter 1226, an algorithm filter 1227, a task constructor 1228, a task allocator 1229, a processing status reader 1230, a status data parser 1231, a task database 1232, etc. It should be noted that the gateway device 122 is provided for illustration purposes, and is not intended to limit the scope of the present disclosure.

The request receiver 1221 may obtain a request for analyzing target image data stored in an image management platform 120 (e.g., an image database 124) from the image management platform 120 (or a user terminal 110). The request result retriever 1222 may determine whether the target image data has been analyzed, for example, by determining whether the task database 1232 (or a storage device of the image management platform 120) includes a historical image analysis task corresponding to the target image data. If the task database 1232 includes a historical image analysis task corresponding to the target image data, the request result retriever 1222 may obtain an analysis abstract and/or an analysis status corresponding to the historical image analysis task. If the task database 1232 includes no image analysis task corresponding to the request, a new image analysis task corresponding to the request may be generated. The image data retriever 1223 may obtain the target image data and information (e.g., one or more features, user permission, etc.) of the target image data from the image database 124. The data availability checker 1224 may determine the availability of the target image data by determining whether the one or more features of the target image data satisfy a first condition. If the one or more features of the target image data satisfy the first condition, the privacy and authorization filter 1225 may determine whether user permission corresponding to the request satisfies a second condition. If the user permission corresponding to the request satisfies the second condition, the custom rule filter 1226 may determine whether the request satisfies a custom rule. If the request satisfies the custom rule, the algorithm filter 1227 may determine a target algorithm from a plurality of candidate algorithms based on the target image data. Accordingly, the task constructor 1228 may generate the image analysis task corresponding to the request. The task allocator 1229 may allocate the image analysis task to a target analyzing device corresponding to the target algorithm. That is, the gateway device 122 may transmit the image analysis task to the image analysis service platform 130.

In some embodiments, before the image analysis task is transmitted to the image analysis service platform 130, the gateway device 122 may determine whether a remaining resource of the target analyzing device is enough for running the image analysis task by using the processing status reader 1230 and the status data parser 1231 to process information of the target analyzing device.

FIG. 8 is a block diagram illustrating an exemplary image analysis service platform 130 according to some embodiments of the present disclosure. In some embodiments, the image analysis service platform 130 may be in communication with a computer-readable storage medium and the modules of the image analysis service platform 130 may execute instructions stored in the computer-readable storage medium.

As illustrated in FIG. 8, the image analysis service platform 130 may include an obtaining module 810 and an analysis module 820.

The obtaining module 810 may be configured to obtain an image analysis task with respect to target image data. The image analysis task may include a storage address of the target image data in an image management platform. More descriptions regarding the obtaining of the image analysis task may be found elsewhere in the present disclosure. See, e.g., operation 902 and relevant descriptions thereof.

The obtaining module 810 may be configured to acquire the target image data from the storage address. More descriptions regarding the obtaining of the target image data may be found elsewhere in the present disclosure. See, e.g., operation 904 and relevant descriptions thereof.

The analysis module 820 may be configured to perform image analysis on the target image data. For example, the analysis module 820 may retrieve a target analyzing device corresponding to a target algorithm based on the image analysis task, and direct the target analyzing device to perform the image analysis on the target image data using the target algorithm. More descriptions regarding the analysis of the target image data may be found elsewhere in the present disclosure. See, e.g., operation 906 and relevant descriptions thereof.

It should be noted that the above descriptions of the image analysis service platform 130 are provided for the purposes of illustration, and are not intended to limit the scope of the present disclosure. For persons having ordinary skills in the art, various variations and modifications may be conducted under the guidance of the present disclosure. However, those variations and modifications do not depart from the scope of the present disclosure. In some embodiments, the image analysis service platform 130 may include one or more other modules. For example, the image analysis service platform 130 may include a storage module to store data generated by the modules in the image analysis service platform 130. In some embodiments, any two of the modules may be combined as a single module, and any one of the modules may be divided into two or more units.

FIG. 9 is a flowchart illustrating an exemplary process 900 for image analysis according to some embodiments of the present disclosure. In some embodiments, the process 900 may be implemented in the image analysis service platform 130 illustrated in FIG. 8. For example, the process 900 may be stored in a storage device (e.g., a storage device of the image analysis service platform 130, an external storage device) in the form of instructions (e.g., an application), and invoked and/or executed by the image analysis service platform 130.

In 902, the image analysis service platform 130 (e.g., the obtaining module 810) may obtain an image analysis task with respect to target image data. The image analysis task may include a storage address of the target image data in an image management platform.

The image analysis task may be used to direct an image analysis service platform to analyze the target image data and include rules or requirements specifying how to analyze the target image data. More descriptions regarding the image analysis task may be found elsewhere in the present disclosure (e.g., FIGS. 3-6 and the descriptions thereof).

In some embodiments, the gateway device 122 may transmit the image analysis task to the image analysis service platform 130, and the image analysis service platform 130 may obtain the image analysis task.

In 904, in response to the image analysis task, the image analysis service platform 130 (e.g., the obtaining module 810) may acquire the target image data from the storage address.

For example, the image analysis service platform 130 may acquire the target image data from the image management platform (e.g., the image database 124) based on the storage address of the target image data in the image management platform.

In 906, the image analysis service platform 130 (e.g., the analysis module 820) may perform image analysis on the target image data.

For example, the image analysis service platform 130 may retrieve a target analyzing device corresponding to a target algorithm based on the image analysis task (e.g., an identification of the target analyzing device), and direct the target analyzing device to perform the image analysis on the target image data using the target algorithm.

In some embodiments, after the image analysis is performed on the target image data, the image analysis service platform 130 may generate an image analysis report of the target image data. The image analysis report may include an analysis abstract, an analysis status, an image analysis result, or the like, or any combination thereof. More descriptions regarding the image analysis report may be found elsewhere in the present disclosure (e.g., FIG. 3 and the descriptions thereof).

In some embodiments, the image analysis service platform 130 may transmit the image analysis report to the gateway device 122 (or the image management platform 120) and/or the user terminal 110. For example, the gateway device 122 and/or the user terminal 110 may send a second request for obtaining the image analysis result of the target image data. In response to the second request, the image analysis service platform 130 may transmit the image analysis report to the gateway device 122 and/or the user terminal 110.

In some embodiments, the image analysis task may include a storage time period. After the image analysis task is performed and the storage time period is reached, the image analysis service platform 130 may delete the target image data. By deleting the target image data, information leakage due to a permanent storage of the target image data on a third-party platform (e.g., the image analysis service platform 130) may be avoided, which can improve the privacy and security of the target image data.

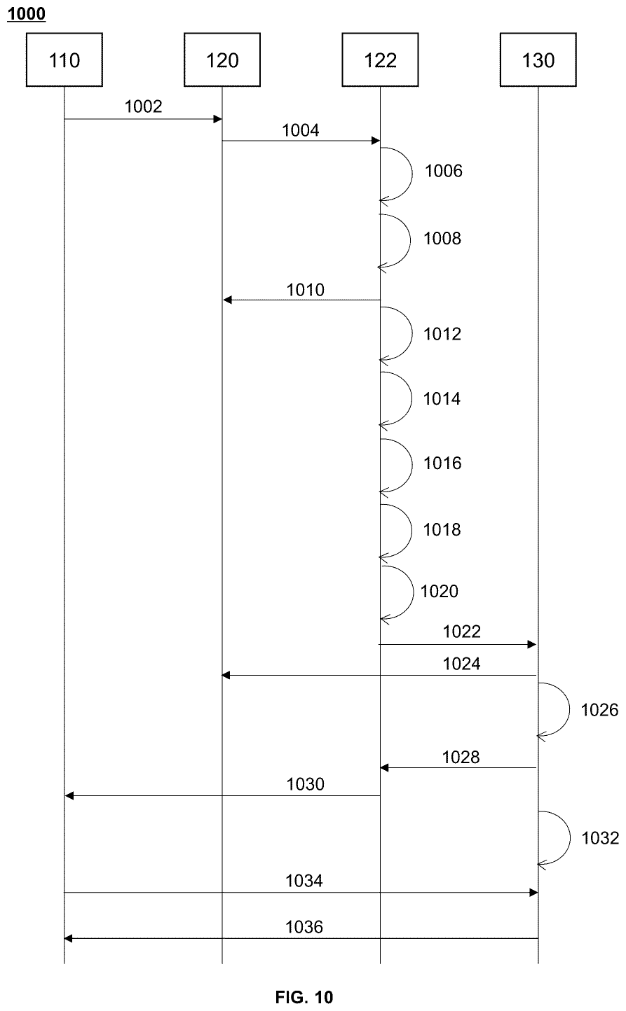

FIG. 10 is a flowchart illustrating an exemplary process 1000 for image analysis according to some embodiments of the present disclosure.

In 1002, the user terminal 110 may send, to the image management platform 120, a request for analyzing target image data stored in the image management platform 120.

In 1004, the gateway device 122 may obtain, from the image management platform 120, the request for analyzing the target image data stored in the image management platform 120.

In 1006, the gateway device 122 may determine whether the target image data has been analyzed, for example, by determining whether a task database (or a storage device of the image management platform 120) includes a historical image analysis task corresponding to the target image data.

In 1008, if the task database includes a historical image analysis task corresponding to the target image data, the gateway device 122 may obtain an analysis abstract and/or an analysis status corresponding to the historical image analysis task.

In 1010, if the task database includes no image analysis task corresponding to the request, the gateway device 122 may obtain the target image data and information (e.g., one or more features, user permission, etc.) of the target image data from the image management platform 120 (e.g., the image database 124).

In 1012, the gateway device 122 may determine the availability of the target image data by determining whether the one or more features of the target image data satisfy a first condition.

In 1014, if the one or more features of the target image data satisfy the first condition, the gateway device 122 may determine whether user permission corresponding to the request satisfies a second condition.

In 1016, if the user permission corresponding to the request satisfies the second condition, the gateway device 122 may determine a target algorithm from a plurality of candidate algorithms based on the target image data.

In 1018, the gateway device 122 may generate the image analysis task

corresponding to the request. The image analysis task may include a storage address of the target image data in the image management platform 120.

In 1020, the gateway device 122 may determine whether the image analysis service platform is available.

In 1022, if the image analysis service platform is available, the gateway device 122 may transmit the image analysis task to the image analysis service platform 130.

In 1024, the image analysis service platform 130 may acquire the target image data from the storage address.

In 1026, the image analysis service platform 130 may perform image analysis on the target image data to generate an image analysis report of the target image data. The image analysis report may include an analysis abstract, an analysis status, an image analysis result, etc.

In 1028, the gateway device 122 may obtain the image analysis report of the target image data from the image analysis service platform 130.

In 1030, the gateway device 122 may transmit the analysis abstract and/or the analysis status to the user terminal 110.

In 1032, the image analysis service platform 130 may delete the target image data.

In 1034, the user terminal 110 may send, to the image analysis service platform 130, a second request for obtaining the image analysis result of the target image data.

In 1036, the user terminal 110 may receive the image analysis result from the image analysis service platform 130, and display the image analysis result.

Processes 300-600, 900, and 1000 may be implemented in the image analysis system 100 illustrated in FIG. 1. For example, the processes 300-600, 900, and 1000 may be stored in a storage device in the form of instructions (e.g., an application), and invoked and/or executed by the gateway device 122 and/or the image analysis service platform 130. The operations of the illustrated process presented below are intended to be illustrative. In some embodiments, the processes 300-600, 900, and 1000 may be accomplished with one or more additional operations not described, and/or without one or more of the operations discussed. Additionally, the order in which the operations of the processes 300-600, 900, and 1000 as illustrated in FIGS. 3-6, 9, and 10 are not intended to be limiting.

It should be noted that the descriptions of the processes 300-600, 900, and 1000 are provided for the purposes of illustration, and are not intended to limit the scope of the present disclosure. For persons having ordinary skills in the art, various variations and modifications may be conducted under the teaching of the present disclosure. However, those variations and modifications may not depart from the protection of the present disclosure.

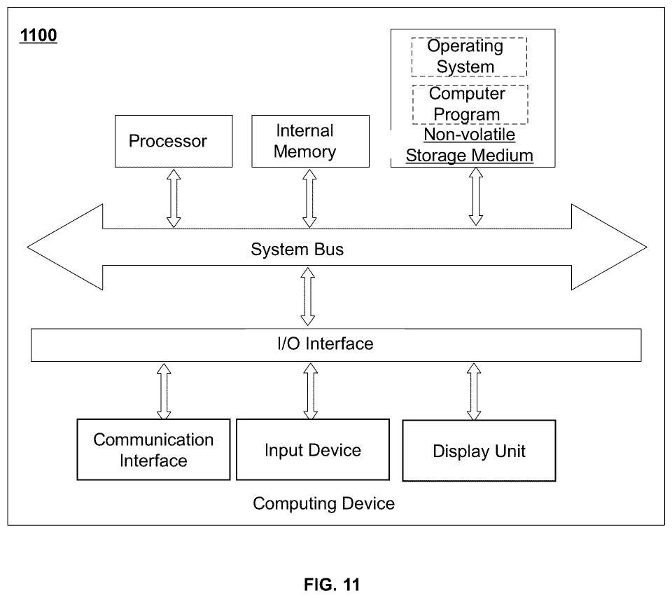

FIG. 11 is a schematic diagram illustrating an exemplary computing device 1100 according to some embodiments of the present disclosure.

In some embodiments, one or more components of the image analysis system 100 may be implemented on the computing device 1100. For example, a processing engine may be implemented on the computing device 1100 and configured to implement the functions and/or methods disclosed in the present disclosure.

The computing device 1100 may include any components used to implement the image analysis system 100 described in the present disclosure. For example, the gateway device 122 and/or the image analysis service platform 130 may be implemented through hardware, software programs, firmware, or any combination thereof, on the computing device 1100. For illustration purposes, only one computer is described in FIG. 11, but computing functions related to the image analysis system 100 described in the present disclosure may be implemented in a distributed fashion by a group of similar platforms to spread the processing load of the image analysis system 100.

The computing device 1100 may include a communication port connected to a network to achieve data communication. The computing device 1100 may include a processor (e.g., a central processing unit (CPU)), a memory, a communication interface, a display unit, and an input device connected by a system bus. The processor of the computing device 1100 may be used to provide computing and control capabilities. The memory of the computing device 1100 may include a non-volatile storage medium, an internal memory. The non-volatile storage medium may store an operating system and a computer program. The internal memory may provide an environment for the execution of the operating system and the computer program in the non-volatile storage medium. The communication interface of the computing device 1100 may be used for wired or wireless communication with an external terminal. The wireless communication may be realized through Wi-Fi, a mobile cellular network, a near field communication (NFC), etc. When the computer program is executed by the processor, a method for determining feature points may be implemented. The display unit of the computing device 1100 may include a liquid crystal display screen or an electronic ink display screen. The input device of the computing device 1100 may include a touch layer covered on the display unit, a device (e.g., a button, a trackball, a touchpad, etc.) set on the housing of the computing device 1100, an external keyboard, an external trackpad, an external mouse, etc.

Merely for illustration, only one processor is described in FIG. 11. However, it should be noted that the computing device 1100 in the present disclosure may also include multiple processors. Thus operations and/or method steps that are performed by one processor as described in the present disclosure may also be jointly or separately performed by the multiple processors. For example, if the processor of the computing device 1100 in the present disclosure executes both operation A and operation B, it should be understood that operation A and operation B may also be performed by two or more different processors jointly or separately (e.g., a first processor executes operation A and a second processor executes operation B, or the first and second processors jointly execute operations A and B).

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended for those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate +20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A method, implemented by a gateway device between an image management platform and an image analysis service platform, the method comprising:
obtaining a request for analyzing target image data stored in the image management platform;
in response to the request, generating an image analysis task with respect to the target image data, the image analysis task including a storage address of the target image data in the image management platform; and
transmitting the image analysis task to the image analysis service platform to direct the image analysis service platform to acquire the target image data from the storage address and perform image analysis on the target image data.

2. The method of claim 1, wherein the generating an image analysis task with respect to the target image data includes:
determining whether one or more features of the target image data satisfy a first condition; and
in response to determining that the one or more features of the target image data satisfy the first condition, generating the image analysis task with respect to the target image data.

3. The method of claim 1, wherein the generating an image analysis task with respect to the target image data includes:
determining whether user permission corresponding to the request satisfies a second condition; and
in response to determining that the user permission corresponding to the request satisfies the second condition, generating the image analysis task with respect to the target image data.

4. The method of claim 1, wherein the generating an image analysis task with respect to the target image data includes:
obtaining a plurality of candidate algorithms;
determining a target algorithm from the plurality of candidate algorithms based on the target image data; and
generating the image analysis task with respect to the target image data based on the target algorithm.

5. The method of claim 4, wherein the determining a target algorithm from the plurality of candidate algorithms based on the target image data includes:
for each of the plurality of candidate algorithms, determining at least one mandatory requirement and at least one optional requirement with respect to an input of the candidate algorithm;
selecting at least one candidate algorithm from the plurality of candidate algorithms, the target image data satisfying the at least one mandatory requirement of each selected candidate algorithm; and
determining the target algorithm from the at least one selected candidate algorithm based on the target image data and the at least one optional requirement of each selected candidate algorithm.

6. The method of claim 4, wherein the determining a target algorithm from the plurality of candidate algorithms based on the target image data includes:
obtaining a corresponding relationship between the plurality of candidate algorithms and a plurality of reference feature vectors representing a plurality of sets of reference image data;
determining a target feature vector representing the target image data; and
determining the target algorithm from the plurality of candidate algorithms based on the target feature vector and the corresponding relationship.

7. The method of claim 4, wherein the determining a target algorithm from the plurality of candidate algorithms based on the target image data includes:
obtaining an algorithm determination model, wherein the algorithm determination model is a trained machine learning model; and
determining the target algorithm based on the target image data using the algorithm determination model.

8. The method of claim 1, further comprising:
obtaining, from the image analysis service platform, an analysis abstract of an image analysis result of the target image data; and
storing the analysis abstract into a storage component of the gateway device.

9. The method of claim 1, further comprising:
obtaining, from a user terminal, a second request for obtaining an image analysis result of the target image data; and
transmitting the second request to the image analysis service platform to direct the image analysis service platform to transmit the image analysis result to the user terminal.

10. The method of claim 1, wherein the gateway device is integrated into the image management platform.

11. A system, including a gateway device between an image management platform and an image analysis service platform, comprising:
at least one storage device including a set of instructions; and
at least one processor configured to communicate with the at least one storage device, wherein when executing the set of instructions, the at least one processor is configured to direct the system to perform operations including:
obtaining a request for analyzing target image data stored in the image management platform;
in response to the request, generating an image analysis task with respect to the target image data, the image analysis task including a storage address of the target image data in the image management platform; and
transmitting the image analysis task to the image analysis service platform to direct the image analysis service platform to acquire the target image data from the storage address and perform image analysis on the target image data.

12. The system of claim 11, wherein the generating an image analysis task with respect to the target image data includes:
determining whether one or more features of the target image data satisfy a first condition; and
in response to determining that the one or more features of the target image data satisfy the first condition, generating the image analysis task with respect to the target image data.

13. The method of claim 11, wherein the generating an image analysis task with respect to the target image data includes:
determining whether user permission corresponding to the request satisfies a second condition; and
in response to determining that the user permission corresponding to the request satisfies the second condition, generating the image analysis task with respect to the target image data.

14. The method of claim 11, wherein the generating an image analysis task with respect to the target image data includes:
obtaining a plurality of candidate algorithms;
determining a target algorithm from the plurality of candidate algorithms based on the target image data; and
generating the image analysis task with respect to the target image data based on the target algorithm.

15-17. (canceled)

18. The method of claim 11, further comprising:
obtaining, from the image analysis service platform, an analysis abstract of an image analysis result of the target image data; and
storing the analysis abstract into a storage component of the gateway device.

19. The method of claim 11, further comprising:
obtaining, from a user terminal, a second request for obtaining an image analysis result of the target image data; and
transmitting the second request to the image analysis service platform to direct the image analysis service platform to transmit the image analysis result to the user terminal.

20-22. (canceled)

23. A method, implemented by an image analysis service platform, the method comprising:
obtaining an image analysis task with respect to target image data, the image analysis task including a storage address of the target image data in an image management platform;
in response to the image analysis task, acquiring the target image data from the storage address; and
performing image analysis on the target image data.

24. The method of claim 23, wherein the image analysis task with respect to the target image data is transmitted from a gateway device between the image management platform and the image analysis service platform.

25. The method of claim 23, further comprising:
generating an analysis abstract of an image analysis result of the target image data; and
transmitting the analysis abstract to a gateway device.

26. The method of claim 23, further comprising:
obtaining, from a user terminal or the gateway device, a request for obtaining an image analysis result of the target image data; and
transmitting the image analysis result to the user terminal.

27-29. (canceled)
